(19) United States
(12) Patent Application Publication
(10) Pub. No.: US 2014/0017625 A1
(43) Pub. Date: Jan. 16, 2014

(54) INTRA-ORAL CAMERA HAVING A LIQUID LENS FOR IMAGE STABILIZATION

(71) Applicants: Zhaohua Liu, YangPu (CN); Jean-Marc Inglese, Bussy Saint Georges (FR); Tan Wang, PuDong (CN); Xiaogin Yu, Shanghai (CN); Jiwu Zhang, PuDong (CN)

(72) Inventors: Zhaohua Liu, YangPu (CN); Jean-Marc Inglese, Bussy Saint Georges (FR); Tan Wang, PuDong (CN); Xiaogin Yu, Shanghai (CN); Jiwu Zhang, PuDong (CN)

(21) Appl. No.: 14/003,389

(22) PCT Filed: Apr. 8, 2011
(86) PCT No.: PCT/CN2011/000606

§ 371 Date: Sep. 24, 2013

(51) Int. Cl. A61B 5/00

(52) U.S. Cl. CPC A61B 5/0088
USPC 433/29; 433/215

(57) ABSTRACT

An intra-oral camera (10) includes an imaging system (12), an imaging sensor (16) and an image stabilization apparatus (14) comprising a liquid lens (36), lens driver elements (38), a microprocessor (34) and a motion sensor (32). The motion sensor (32) provides a motion signal indicating camera movement. The adjustable liquid lens (36) has an interface between first and second immiscible liquids and is actuable to change refraction with respect to a first axis in response to a first adjustment signal at a first pair of electrodes and with respect to a second axis in response to a second adjustment signal at a second pair of electrodes. The microprocessor (34) is responsive to stored instructions for obtaining the motion signal from the motion sensor (32) and in communication with lens driver elements (38) for providing the first and second adjustment signals to the adjustable liquid lens (36).

### FIELD OF THE INVENTION

The invention relates generally to the field of medical diagnostic instruments, and in particular to an apparatus for dental imaging. More specifically, the invention relates to an intra-oral camera having a liquid lens using a multi-electrode design to provide image stabilization.

### BACKGROUND OF THE INVENTION

While there have been improvements in detection, treatment and prevention techniques, dental caries remains a prevalent condition affecting people of all age groups. If not properly and promptly treated, caries could lead to permanent tooth damage and even to loss of teeth. Thus dental imaging based on an intra-oral camera is of great interest.

There exist known intra-oral cameras, such as those available from ACTEON Inc. of Mount Laurel, N.J., USA. Generally, intra-oral cameras are operated over a large working distance range that typically varies between about 1 mm to about 50 mm. They may have a sizable depth of field (DOF), which is different at different working distances. Thus, focus adjustment is used to provide good image quality. However, for most of the known intra-oral cameras including the one disclosed in U.S. Pat. No. 6,019,721 (Holmes), focus adjustment is performed manually by operator adjustment to the distance between a lens and an imaging sensor. Conventional intra-oral cameras do not have auto-focusing capability and focus must be separately adjusted for each image. Therefore, they are not convenient to use.

Conventional intra-oral cameras, like other cameras, can suffer from image blur due to vibration during image capture. Image stabilization methods aimed at reducing image blur have been proposed. For example, see U.S. Pat. Nos. 4,998,809 (Tsuji) and 5,040,881 (Tsuji) which use a floating optical element.

U.S. Publication No. 2009/0141352 (Jannard) discloses a lens system that employs four liquid lens cells, one pair of liquid lens cells to provide image stabilization in one direction, and another pair of liquid lens cells to provide image stabilization in another direction.

While conventional cameras that provide image stabilization may have achieved certain degrees of success in their particular applications, they fail to meet the dimensional and operational requirements of intra-oral imaging. Thus, there is a need for an intra-oral camera that is small in width, convenient to use, and capable of providing image stabilization in all directions.

### SUMMARY OF THE INVENTION

An object of the present invention is to provide an intra-oral camera capable of providing image stabilization in at least two orthogonal directions.

Another object of the present invention is to provide an intra-oral camera comprising a liquid lens that uses a multi-electrode design to provide image stabilization in at least two orthogonal directions.

A further object of the present invention is to provide an intra-oral camera consisting of only one liquid lens that uses a multi-electrode design to provide image stabilization in at least two orthogonal directions.

The advantages of the present invention are that the inventive camera is small in its width, large in its length to width ratio, convenient in use, and capable of reducing image blur during image capturing in the mouth of a patient.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to one aspect of the present invention, there is provided an intra-oral camera comprising: an imaging system comprising an imaging sensor and one or more light-directing elements that direct light along an optical path to the imaging sensor; and an image stabilization apparatus for adjusting light direction along the optical path to compensate camera movement and comprising: (i) a motion sensor that provides a motion signal that is indicative of camera movement; (ii) an adjustable liquid lens disposed along the optical path and comprising an interface between first and second immiscible liquids, wherein the adjustable liquid lens is actuable to change refraction with respect to a first axis in response to a first adjustment signal at a first pair of electrodes and with respect to a second axis in response to a second adjustment signal at a second pair of electrodes, wherein the first and second axes are orthogonal with respect to each other and are both orthogonal with respect to the optical path; (iii) a microprocessor responsive to stored instructions for obtaining the motion signal from the motion sensor and in communication with a plurality of lens driver elements for providing the first and second adjustment signals to the adjustable liquid lens.

According to another aspect of the invention, there is provided an intra-oral camera comprising: an imaging system comprising an imaging sensor and one or more light-directing elements that direct light along an optical path to the imaging sensor; and an image stabilization system for adjusting light direction along the optical path comprising a motion sensor, a microprocessor in communication with the motion sensor, a liquid lens having two pairs of electrodes, and four liquid lens drivers in communication with the microprocessor for applying adjustment signals to the electrodes to drive the liquid lens, wherein the liquid lens includes a vessel filled with a first liquid having a first optical index and a second liquid, immiscible with the first liquid and having a second optical index, wherein the second liquid is in contact with the first liquid along an interface, wherein the first and second liquids are of substantially the same density, and wherein the respective first and second optical indices differ from each other, and wherein the voltage at the pairs of electrodes adjusts the tilt of an optical axis in a first direction that is perpendicular to the optical axis, in a second direction that is perpendicular to the first direction and to the optical axis, and in a combination of the first and second directions for stabilizing the image formed on the imaging sensor.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of an intra-oral camera of the present invention.

FIG. 2A shows a comparative auto-focusing system using a liquid lens having two electrodes.

FIG. 2B shows an image stabilization system according to the present invention.

FIG. 3A shows a liquid lens having two electrodes when the voltage is zero.

FIG. 3B shows the two-electrode liquid lens used when the voltage is not zero.

FIG. 3C shows the working principle of the two-electrode liquid lens.

FIG. 4A shows a front view of a liquid lens having four electrodes used in the present invention.

FIG. 4B shows a side view of the four-electrode liquid lens in the x-z plane when the optical axis of the liquid lens is in the direction of the optical axis of the imaging system.

FIG. 4C shows a side view of the four-electrode liquid lens when the optical axis of the liquid lens is tilted in the x-z plane.

FIG. 4D shows a side view of the four-electrode liquid lens in the y-z plane when the optical axis of the liquid lens is in the direction of the optical axis of the imaging system.

FIG. 4E shows a side view of the four-electrode liquid lens when the optical axis of the liquid lens is tilted in the y-z plane.

### DETAILED DESCRIPTION OF THE INVENTION

The following is a detailed description of the preferred embodiments of the invention, reference being made to the drawings in which the same reference numerals identify the same elements of structure in each of the several figures.

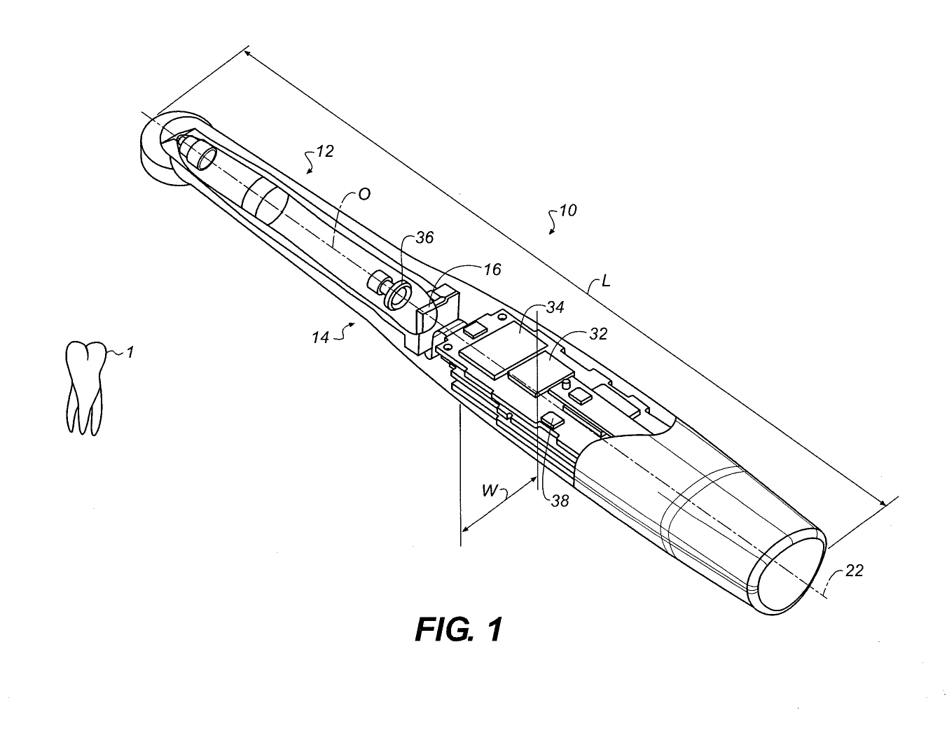

FIG. 1 shows an intra-oral camera 10 of the present invention according to one embodiment. Intra-oral camera 10 comprises an illumination system 11 (not shown), an imaging system 12, an image stabilization apparatus 14, and an imaging sensor 16. Intra-oral camera 10 has a width W and a length L, the width and the length being perpendicular and parallel to the axial direction 22, respectively. Image stabilization system 14 includes a liquid lens 36 having multiple electrodes, liquid lens drivers 38, a microprocessor 34, and a motion sensor 32.

Intra-oral camera 10 is intended for imaging a target 1 that is within the mouth of a patient, and to do this expediently and accurately. Target 1 can be a tooth, for example.

Imaging system 12 comprises a lens or a group of lenses as light-directing elements that direct light along an optical path O and that provide a large depth of field (DOF). Design of such a lens system is familiar to those skilled in the optical design arts. In one embodiment, imaging system 12 comprises three lens groups as light-directing elements. In one embodiment, each lens in imaging system 12 is located at a fixed position along the optical path O. In operation, imaging system 12 images target 1 onto imaging sensor 16, located at a fixed imaging plane.

For intra-oral use, the width W of intra-oral camera 10 is preferably not more than about 35 mm, more preferably not more than about 30 mm, and most preferably not more than about 25 mm. The length to width ratio, defined as L/W, is between 3 and 12, and more preferably between 5 and 8. Additionally, the working distance of intra-oral camera 10 is between about 1 and 300 mm. These requirements for a large length to width ratio, narrow width, and specific working distance are needed in order to fit the camera comfortably within the mouth of the patient. In embodiments of the present invention, these requirements are met by using an adjustable liquid lens having four or more electrodes. The liquid lens that is used provides an adjustable lens element disposed along the optical path and actuable to change refraction with respect to each of two orthogonal axes in response to received adjustment signals. The use of this type of adjustable lens differentiates intra-oral camera 10 of the present invention from conventional intra-oral cameras and from many other types of conventional cameras that are intended for other uses.

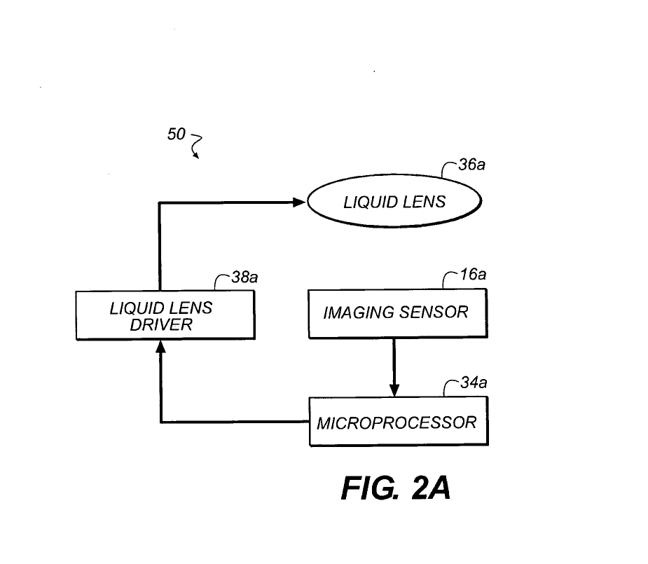

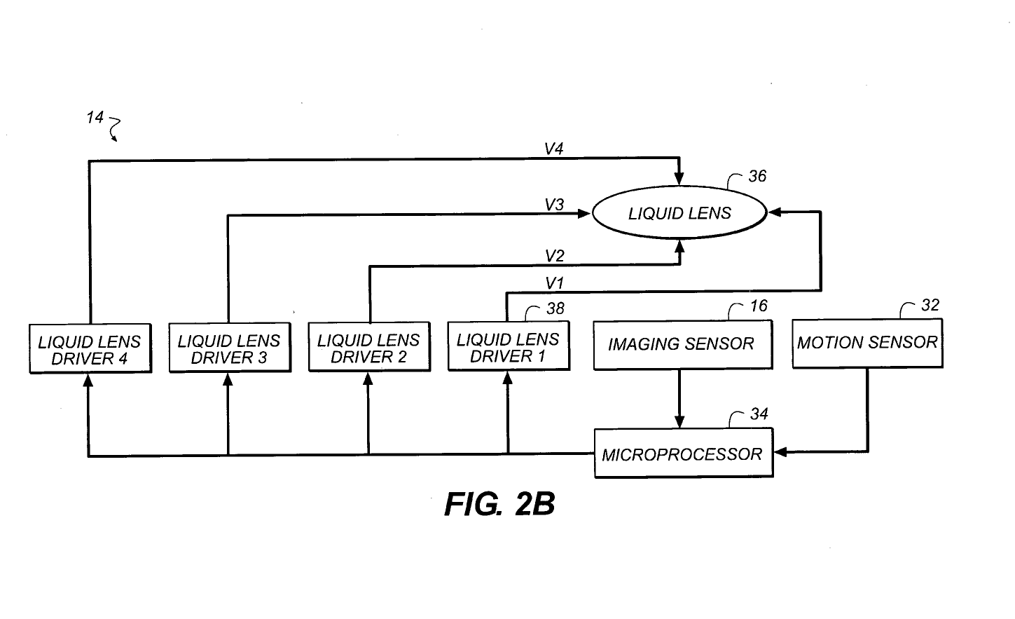

FIG. 2A shows a comparative auto-focusing system 50 using a liquid lens 36a having two electrodes. Auto-focusing system 50 comprises an imaging sensor 16a, a microprocessor 34a, a liquid lens driver 38a and liquid lens 36a. In this system, imaging sensor 16a transmits an image signal to microprocessor 34a, which then analyzes the image signal, generates a voltage signal, and sends the voltage signal to liquid lens driver 38a. Liquid lens driver 38a then applies a proper level of voltage on liquid lens 36a.

FIG. 2B shows image stabilization system 14 comprising motion sensor 32, microprocessor 34, liquid lens 36, and liquid lens drivers 38. Liquid lens 36 is located between imaging system 12 and imaging sensor 16. Liquid lens 36 is an electro-wetting type, such as a liquid lens available from Varioptic (Lyon, France).

Motion sensor 32 is a gyroscope in one example. The gyroscope measures or maintains orientation based on the principle of conservation of angular momentum. Motion sensor 32 can use a spinning wheel or disk whose axle is free to take any angular orientation. Due to conservation of angular momentum, this angular orientation changes much less in response to a given external torque than it would without the large angular momentum associated with the gyroscope's high rate of spin. The gyroscope is mounted in gimbals to minimize the external torque. Consequently, the orientation of the gyroscope remains nearly fixed, regardless of any motion of the platform on which it is mounted. Therefore, the gyroscope is advantageous for use as a motion detector to detect the movement of intra-oral camera 10 and send the movement information to microprocessor 34.

Optionally, imaging sensor 16 may also provide movement information to microprocessor 34 because image quality characteristics such as image sharpness also reflect the vibration of intra-oral camera 10. Generally, however, the movement information from imaging sensor 16 alone may not be adequate for image stabilization. However, when combined with the movement information provided by motion sensor 32, the movement information from imaging sensor 16 can also be useful.

Motion sensor 32 provides a motion signal indicative of camera movement. Microprocessor 34 is responsive to stored instructions for obtaining the motion signal from the motion sensor and is in communication with a plurality of lens driver elements for providing the first and second adjustment signals to the adjustable liquid lens element. Microprocessor 34 analyzes the movement information from this motion signal, converts it into a voltage signal that corresponds to four or more of a plurality of voltages, and determines where to best apply these voltages to liquid lens 36 through respective liquid lens driver elements 38. In special cases, two or more of the applied voltages may be equal. Microprocessor 34 also transmits the voltage signal to liquid lens driver elements 38. In one example, microprocessor 34 transmits the voltage signal to four liquid lens drivers (Liquid lens driver 1-Liquid lens driver 4), which then apply four adjustment signals, voltages V1, V2, V3, and V4, to liquid lens 36. When the liquid lens drivers apply the voltages as adjustment signals to liquid lens 36, the shape of the liquid interface between first and second liquids in liquid lens 36 changes. This change in the shape of the liquid interface at liquid lens 36 also helps to compensate for movement of other lenses in imaging system 12 with respect to the optical path so that target 1 is imaged on imaging sensor 16 with good focus when intra-oral camera 10 is moved or vibrated.

Alternatively, the four liquid lens drivers can be replaced with a single special liquid lens driver that can provide four independent voltages as adjustment signals.

One feature of this inventive intra-oral camera is that liquid lens 36 uses a multi-electrode design, meaning that liquid lens 36 has four or more electrodes and is thus actuable to stabilize the image along two orthogonal axes in response to received adjustment signals. As a comparison, a conventional liquid lens uses two electrodes and, at best, is actuable to stabilize the image only along a single axis in response to received adjustment signal.

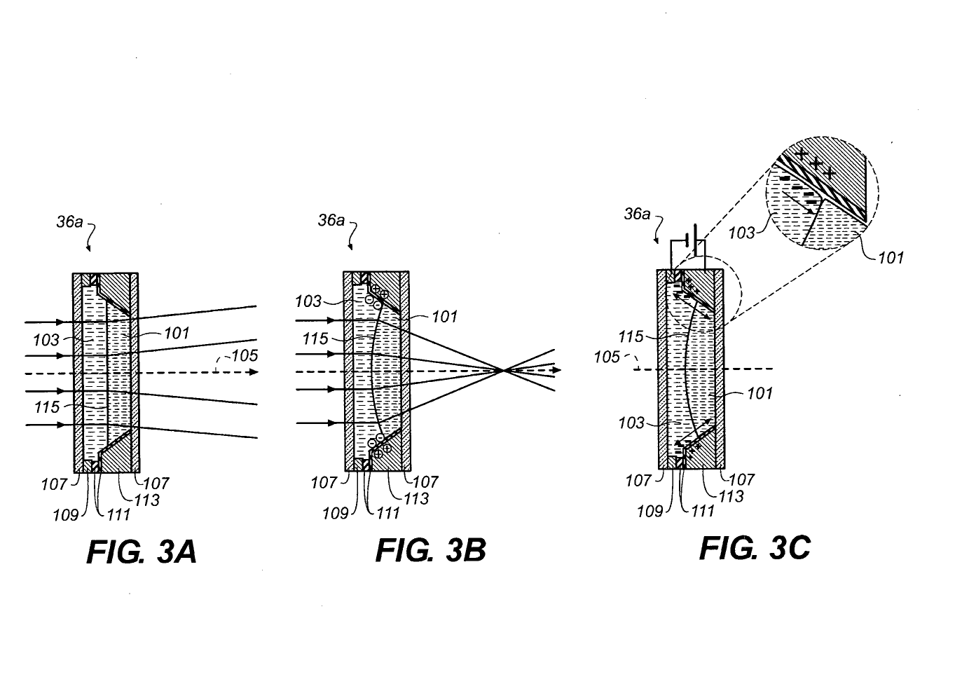

In order to better understand how embodiments of the present invention are advantaged over camera embodiments using conventional liquid lenses, it is useful to review how the liquid lens operates and to understand differences between liquid lenses that use two electrodes, that is, a single pair of electrodes, and lenses using four electrodes, that is, two pairs of electrodes. Referring to FIGS. 3A-3C, which correspond to FIG. 1 and FIG. 2 of WO 2010/057336 (Liu), a conventional liquid lens 36a having two electrodes also generally includes two kinds of liquids of equal density. The liquids are sandwiched between two transparent windows 107 in a conical vessel. In one embodiment, one liquid is conductive water 103, while the other is oil 101 for providing a measure of stability for the optical axis 105. Liquid lens 36a further includes electrodes 109 and 113 insulated from oil 101 but in electrical contact with water 103; and variable voltage can be selectively applied to the electrodes as an adjustment signal. Insulator 111 is deposited between electrodes 109 and 113 to separate them. The interface 115 between oil 101 and water 103 will change its shape depending on the voltage applied across the conical structure. As shown in FIG. 3A, when zero volts are applied, interface 115 is slightly curved and the surface of oil 101 becomes concave. When the voltage is increased to about 40 volts, the surface of oil 101 becomes highly convex, as shown in FIG. 3B. In this way, liquid lens 36a can attain the desired refraction power by means of changing the voltage applied on the electrodes.

FIG. 3C shows the working principle of liquid lens 36a having two electrodes. Liquid lens 36a works based on the electro-wetting phenomenon described below: a drop of water 103 is deposited on a substrate made of metal, covered by a thin insulating layer. The voltage applied to the substrate generates an electrostatic pressure to force the liquid change its shape so as to modify the contact angle of the liquid drop. Two iso-density liquids are employed in the liquid lens: one is an insulator such as oil 101 while the other is a conductor such as water 103. The variation of voltage leads to a change of curvature of the liquid-liquid interface 115, which in turn leads to a change of optical power or refraction of the lens.

In spite of some similarities between liquid lens 36 having four electrodes (two pairs of electrodes) and conventional liquid lens 36a having two electrodes, their operation and capabilities differ significantly. Firstly, the optical axis of liquid lens 36a having two electrodes, as shown in FIG. 3A-3C, cannot be tilted away from its normal direction because the electrodes are arranged symmetrically over the entire interface of the two liquids. Thus a single two-electrode liquid lens cannot be used to provide image stabilization.

When one of the two electrodes in liquid lens 36a′ is separated into two parts and the voltage signal that provides the adjustment signal is applied on two points of the interface of the two liquids (see U.S. Publication No. 2009/0141352 by Jannard), the optical axis of liquid lens 36a can only change with respect to a single axis. Thus, a liquid lens modified in this way can compensate the vibration or other movement of the camera in one direction only. In order to compensate the vibration in two orthogonal directions, two separate liquid lenses, each having two electrodes must be used, as described in the '1352 Jannard application. In contrast, one liquid lens 36 having four separated electrodes, as described with reference to embodiments of the present invention, is sufficient to provide image stabilization in two orthogonal directions that are perpendicular to the optical axis of the imaging system.

A second difference between the capabilities and use of conventional liquid lenses and the liquid lens having two pairs of electrodes relates to driver components. Liquid lens driver 38 for driving four-electrode liquid lens 36 differ from liquid lens drivers 38a for driving the two-electrode liquid lens 36a because liquid lens drivers 38 must provide four different voltages that correspond to the two-dimensional tilt in optical axis of liquid lens 36, while liquid lens driver 38a only provides a single voltage across the liquid lens.

As yet another difference, microprocessor 34 logic for sending voltage signals to liquid lens drivers 38 for driving the four-electrode liquid lens 36 is different from microprocessor 34a logic for sending voltage signals to liquid lens driver 38a to drive the two-electrode liquid lens 36a. This is because, for the four-electrode case, microprocessor 34 must generate four different voltage signals that correspond to the two-dimensional tilt in optical axis of liquid lens 36. Thus, microprocessor 34 uses an algorithm that differs from that in microprocessor 34a.

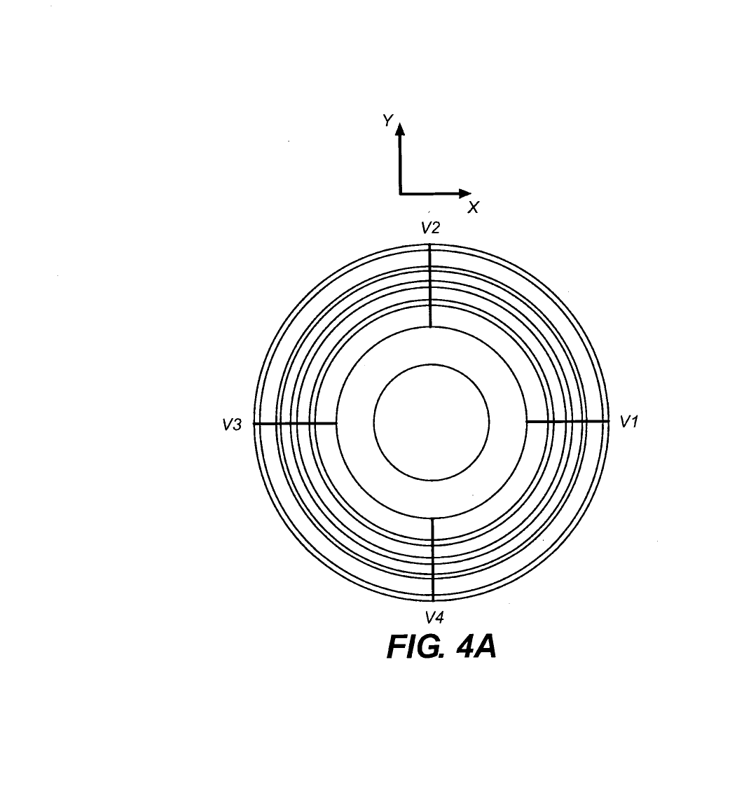

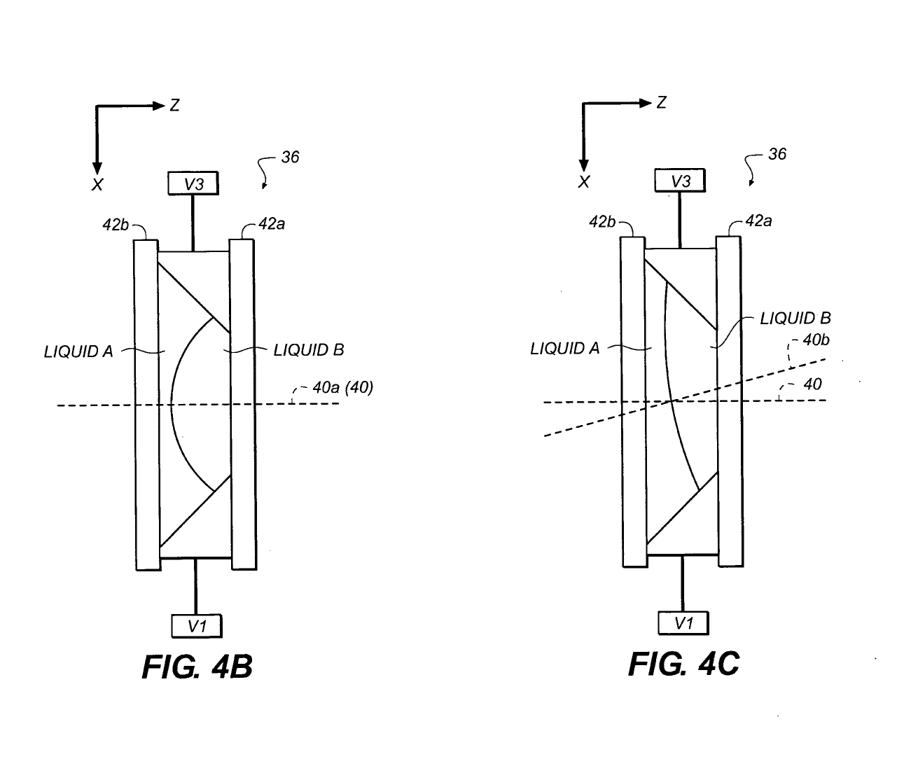

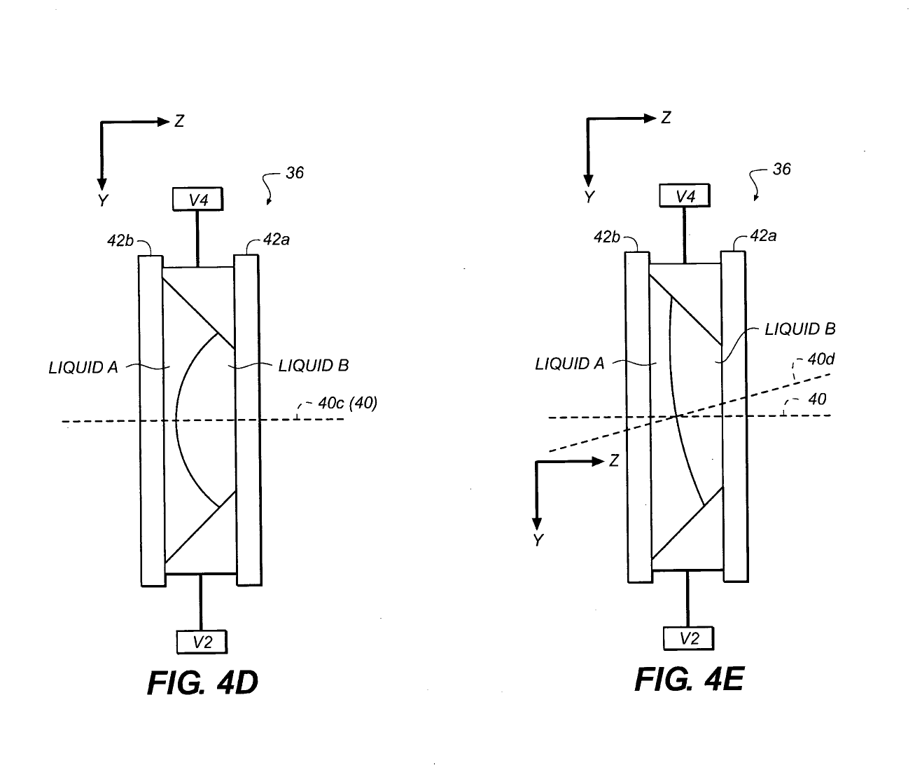

FIG. 4A shows the front view of liquid lens 36 having four electrodes to which four respective voltages V1, V2, V3, and V4 are applied as adjustment signals. For one pair of electrodes, voltages V1 and V3 are used to control the shape of the liquid interface in the x-direction, while voltages V2 and V4 at the other pair of electrodes control the shape in the y-direction.

FIG. 4B and FIG. 4C show side views of liquid lens 36 in the x-z plane when V1 and V3 are equal and when V1 and V3 are unequal, respectively. Liquid lens 36 comprises two substrates 42a, 42b and two liquids—Liquid A and Liquid B. The optical axis 40 of imaging system 12 is parallel to the z-direction, which is perpendicular to the x-y plane.

Generally, Liquid A and Liquid B are immiscible and have different optical indexes. These liquids have substantially equal density, that is, densities preferably within ±12% of each other. One is generally an insulating liquid, for example, comprising oil or an oily substance with a first index of refraction, and the other is typically a conducting liquid comprising for example an aqueous solution having a second index of refraction. These liquids are sandwiched between two transparent substrates 42a, 42b in a conical vessel. In one embodiment, for example, Liquid A is water 103, which is conductive, while encasing Liquid B, an oil 101, acts as a lid. This allows a fixed volume of water to be contained, and provides a measure of stability for the optical axis 40 of liquid lens 36. The relative shape of the interface between liquids determines the refractive properties of the lens. The relative indices of refraction of the two liquids must differ from each other by some amount in order to provide adjustable refraction.

When adjustment signals V1=V3 as shown in FIG. 4B, liquid lens 36 acts as a normal lens in the x-z plane, whose optical axis 40a in the x-z plane is along optic axis 40 of imaging system 12. In this case, liquid lens 36 only provides focus adjustment function in the x-z plane.

FIG. 4C shows that optical axis 40b of liquid lens 36 is tilted in the x-z plane away from optic axis 40 of imaging system 12 when adjustment signals V1 and V3 are not equal. This tilt in the x-z plane is also referred to as a tilt in the x direction because the tilt of optical axis 40b can compensate the vibration or other movement of intra-oral camera 10 in the x-direction.

Likewise, FIG. 4D shows a side view of liquid lens 36 in the y-z plane when adjustment signals V2 and V4 are equal. Liquid lens 36 acts as a normal lens whose optical axis 40c is along optic axis 40 of imaging system 12. In this case, liquid lens 36 only provides a focus adjustment function in the y-z plane.

Similar to FIG. 4C, FIG. 4E shows that optical axis 40d of liquid lens 36 is tilted in the y-z plane away from optic axis 40 of imaging system 12 when adjustment signals V2 and V4 are not equal. This tilt in the y-z plane is also referred to as a tilt in the y direction because the tilt of optical axis 40d can compensate the vibration or other movement of intra-oral camera 10 in the y-direction.

In summary, because liquid lens 36 used in the present invention has four electrodes to which four different voltages can be applied to particular areas of the lens, the shape of the interface of the two liquids in the liquid lens can be selectively tilted in a first direction (in the x-z plane), or in a second direction (in the y-z plane), or in both directions at the same time. As a result, the optical axis of liquid lens 36 can be selectively tilted in the x-z and y-z planes independently. This essentially gives liquid lens 36 the capability to tilt its optical axis to compensate the vibration or other movement of the camera in any direction through combined tilts in the x-z and y-z planes. Thus liquid lens 36 can provide optical image stabilization without relying on mechanical movement of any component.

Illuminating system 11 is configured to direct light from a light source in order to illuminate target 1 for improved imaging at imaging sensor 16. The light source can be one or more light emitting diodes (LEDs) or any other known light source. Illumination system 11 can be integrated into the intra-oral camera 10 package or can be provided from a separate device. An optical fiber or other light guide could be provided for directing illumination toward target 1 from an external light source.

Imaging sensor 16 records the image of target 1 at a fixed position. Imaging sensor 16 can be a complementary metal-oxide-semiconductor (CMOS) device, charge coupled device (CCD), or any other known sensor array type.

Though intra-oral camera 10 of the present invention is designed for imaging an intra-oral target, this device may be used in other suitable applications, particularly where the camera width requirement is fairly constrained, such as for endoscope applications.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. An intra-oral camera comprising:
an imaging system comprising an imaging sensor and one or more light-directing elements that direct light along an optical path to the imaging sensor; and
an image stabilization apparatus for adjusting light direction along the optical path to compensate camera movement, comprising:
(a) a motion sensor that provides a motion signal that is indicative of camera movement;
(b) an adjustable liquid lens disposed along the optical path and comprising an interface between first and second immiscible liquids, wherein the adjustable liquid lens is actuable to change refraction with respect to a first axis in response to a first adjustment signal at a first pair of electrodes and with respect to a second axis in response to a second adjustment signal at a second pair of electrodes, wherein the first and second axes are orthogonal with respect to each other and are both orthogonal with respect to the optical path; and
(c) a microprocessor responsive to stored instructions for obtaining the motion signal from the motion sensor and in communication with a plurality of lens driver elements for providing the first and second adjustment signals to the adjustable liquid lens.

2. The intra-oral camera of claim 1, wherein the motion sensor is a gyroscope.

3. The intra-oral camera of claim 1, wherein the liquid lens comprises more than four electrodes.

4. The intra-oral camera of claim 1 having a working distance between 1 and 300 mm.

5. The intra-oral camera of claim 1 having a width and a length, wherein the length to width ratio is between 5 and 8.

6. The intra-oral camera of claim 1, wherein the liquid lens is disposed at a fixed position along the optical path between the imaging system and the sensor.

7. The intra-oral camera of claim 1, wherein each lens of the imaging system is located at a fixed position along the optical path.

8. The intra-oral camera of claim 1, wherein the imaging sensor is located at a fixed position along the optical path.

9. The intra-oral camera of claim 1, wherein the microprocessor analyzes both the movement information detected by the motion sensor and the movement information detected by the imaging sensor.

10. An intra-oral camera comprising:
an imaging system comprising an imaging sensor and one or more light-directing elements that direct light along an optical path to the imaging sensor; and
an image stabilization system for adjusting light direction along the optical path comprising a motion sensor, a microprocessor in communication with the motion sensor, a liquid lens having two pairs of electrodes, and four liquid lens drivers in communication with the microprocessor for applying adjustment signals to the electrodes to drive the liquid lens,
wherein the liquid lens includes a vessel including a first liquid having a first optical index and a second liquid, immiscible with the first liquid and having a second optical index, wherein the second liquid is in contact with the first liquid along an interface, wherein the first and second liquids are of substantially the same density, and wherein the respective first and second optical indices differ from each other, and wherein the voltage at the pairs of electrodes adjusts the tilt of an optical axis in a first direction that is perpendicular to the optical axis, in a second direction that is perpendicular to the first direction and to the optical axis, and in a combination of the first and second directions for stabilizing the image formed on the imaging sensor.

11. An intra-oral camera comprising:
an imaging system for forming an image of a target onto an imaging sensor; and
an image stabilization system comprising a motion sensor, a microprocessor in communication with the motion sensor, a liquid lens having four electrodes, and a liquid lens driver in communication with the microprocessor for applying adjustment signals to the four electrodes to drive the liquid lens,
wherein the liquid lens includes a vessel including a first liquid having a first optical index and a second liquid, immiscible with the first liquid and having a second optical index, wherein the second liquid is in contact with the first liquid along an interface, wherein the first and second liquids are of substantially the same density, and wherein the respective first and second optical indices differ from each other, and
wherein the motion sensor detects the movement of the camera, the microprocessor analyzes the movement information detected by the motion sensor, converts the movement information into adjustment signals and sends the adjustment signals to the liquid lens driver, the liquid lens driver applies a plurality of adjustment signals to the four electrodes of the liquid lens to change the shape of the interface between the first and second liquids and to selectively tilt the optical axis of the liquid lens in a first direction that is perpendicular to the optical axis, in a second direction that is perpendicular to the first direction and to the optical axis, or in any combined direction formed from the first and second directions for stabilizing the image formed on the imaging sensor.

12. A method for obtaining an intra-oral image of a target comprising:
directing light from the target along an optical path toward an image sensor;
sensing movement of the image sensor relative to the target;
adjusting the optical path according to the sensed movement by changing the shape of the interface of two liquids in a liquid lens along either or both first and second axes, wherein the first and second axes are mutually orthogonal and are both orthogonal with respect to the optical path; and
capturing image data of the target according to the light received along the adjusted optical path.

13. The method of claim 12 wherein sensing movement of the image sensor comprises receiving a signal from a gyroscope.

14. The method of claim 12 wherein changing the shape of the interface in the liquid lens comprises applying variable voltage signals to the lens from one or more liquid lens drivers.

15. The method of claim 12 wherein the liquid lens includes a vessel including a first liquid, and a second liquid being in contact with the first liquid, the first and second liquids being immiscible, of different optical indexes, and of substantially same density.
